(19) United States
(12) Patent Application Publication
(10) Pub. No.: US 2023/0353963 A1
(43) Pub. Date: Nov. 2, 2023

(54) SEMI-IMPLANTABLE HEARING AID

(71) Applicant: THE REGENTS OF THE UNIVERSITY OF CALIFORNIA, Oakland, CA (US)

(72) Inventor: Hamid DJALILIAN, Irvine, CA (US)

(73) Assignee: THE REGENTS OF THE UNIVERSITY OF CALIFORNIA, Oakland, CA (US)

(21) Appl. No.: 18/021,782

(22) PCT Filed: Aug. 20, 2021
(86) PCT No.: PCT/US2021/046942

§ 371 Date: Feb. 16, 2023

Related U.S. Application Data
(60) Provisional application No. 63/068,158, filed on Aug. 20, 2020.

(51) Int. Cl. H04R 25/00; A61N 1/36

(52) U.S. Cl. CPC H04R 25/606; A61N 1/36036; H04R 25/554; H04R 2225/67

(57) ABSTRACT

A totally or partially concealed, partially implantable semi-implantable hearing device includes a replaceable outer ear unit having a means for generating energy responsive to acoustic energy received by the replaceable outer ear unit. The replaceable outer ear unit is adapted to be located inside a subject’s outer ear canal or behind a subject’s ear. The implantable semi-implantable hearing device also includes a fluid-filled tube-shaped device that is placed in the middle ear or mastoid. Characteristically, the fluid-filled tube-shaped device is adapted to receive transferred energy from the replaceable outer ear unit such that energy received by the fluid-filled tube-shaped device is transferred to the inner ear via fluid contained in the fluid-filled tube-shaped device.

### CROSS-REFERENCE TO RELATED APPLICATIONS

This application claims the benefit of U.S. Provisional Application Serial No. 63/068,158 filed Aug. 20, 2020, the disclosure of which is hereby incorporated in its entirety by reference herein.

### TECHNICAL FIELD

In at least one aspect, the present invention is related to an implantable and/or semi-implantable hearing device.

### BACKGROUND

Hearing loss affects approximately 10% of the population in the developed world. There are currently approximately 30 million in the U.S. who have hearing loss. Normally, hearing is improved using a hearing aid that is placed within the ear canal. The hearing aid takes sound and converts it into louder sound, which vibrates the eardrum, which in turn vibrates the ossicles (middle ear bones), and that vibrates the inner ear fluids via the oval window. The hearing organ (cochlea) can also be stimulated via the round window (another membranous window of the inner ear).

Currently, the only implantable device that transmits sound to the inner ear via the round window requires an extensive 2-3 hour surgery.

Accordingly, there is a need for improved implantable semi-implantable hearing devices.

### SUMMARY

In at least one aspect, a semi-implantable hearing device is provided. The semi-implantable hearing device can span or about the tympanic membrane (eardrum) and transmit sound information from an external hearing device in the canal to the inner ear. The implantable portion of this device can be placed in the office with topical anesthesia in a 2-3 minute procedure rather than 2-3 hours and general anesthesia. Advantageously, the semi-implantable hearing device can substantially reduce costs.

In another aspect, the semi-implantable hearing device includes a replaceable outer ear unit having an energy transfer device for generating energy responsive to acoustic energy received by the replaceable outer ear unit. The replaceable outer ear unit is adapted to be located inside a subject’s outer ear canal or behind a subject’s ear. The semi-implantable hearing device also includes a fluid-filled tube-shaped device that is placed in the middle ear or mastoid or other structure proximate to the subject’s ear. Advantageously, the fluid-filled tube-shaped device is adapted to receive transferred energy from the replaceable outer ear unit, wherein energy received by the fluid-filled tube-shaped device is transferred to a subject’s inner ear via fluid contained in the fluid-filled tube-shaped device.

In another aspect, the semi-implantable hearing device can be totally or partially concealed.

In another aspect, the semi-implantable hearing device can be placed in a minimally invasive office procedure without general anesthesia.

In another aspect, the semi-implantable hearing device can provide for direct cochlear mechanical vibration, which produces a higher quality sound than conventional hearing devices.

In another aspect, the replaceable outer ear unit is configured to transfer energy to the fluid-filled tube-shaped device via a light-based transmission and piezoelectric translation.

In another aspect, the replaceable outer ear unit is configured to transfer energy to the fluid-filled tube-shaped device via aligned coils such that a first coil in the replaceable outer ear unit is aligned with a second coil in the fluid-filled tube-shaped device.

In another aspect, the semi-implantable hearing device does not require surgery under general anesthesia to be placed in a subject.

In another aspect, the energy is transferred from the replaceable outer ear unit to the fluid-filled tube-shaped device using electromagnetic stimulation.

In another aspect, energy is transferred from the replaceable outer ear unit to the fluid-filled tube-shaped device (e.g., the actuator) via a radiofrequency stimulation.

In another aspect, energy is transferred from the replaceable outer ear unit to the fluid-filled tube-shaped device via a light-based transmission and piezoelectric translation.

In another aspect, the fluid-filled tube-shaped device includes an attached piezoelectric component.

In another aspect, the fluid-filled tube-shaped device includes an attached photo-responsive device that includes a photostrictive material.

In another aspect, energy is transferred from the replaceable outer ear unit to the fluid-filled tube-shaped device via aligned coils.

In another aspect, the fluid-filled tube-shaped device is secured to a subject by attaching a magnet to the ossicular chain in the ear.

In another aspect, the replaceable outer ear unit is hermetically sealed and implanted in a subject’s body.

In another aspect, the fluid-filled tube-shaped device is configured to make inner ear contact with the round window membrane.

In another aspect, the fluid-filled tube-shaped device is configured to make inner ear contact with the oval window or one or more ossicle.

In another aspect, a fluid-filled tube-shaped device is configured to make inner ear contact i with the inner ear through a surgically or disease-created opening into the inner ear.

In another aspect, the fluid-filled tube-shaped device is configured to make outer contact with the tympanic membrane.

In another aspect, the fluid-filled tube-shaped device is configured to make outer contact is made under the skin of the subject’s ear canal.

In another aspect, the fluid-filled tube-shaped device is configured to make outer contact under the skin behind or above the ear.

In another aspect, the filled tube-shaped is configured to make a rigid inner ear contact.

In another aspect, the filled tube-shaped is configured to make a flexible inner ear contact.

In another aspect, the fluid-filled tube-shaped device is configured to make a rigid outer contact.

In another refinement, the fluid-filled tube-shaped device is configured to make a flexible outer contact.

In another aspect, the fluid-filled tube-shaped device is used for transmitting hearing information where the fluid-filled tube-shaped device can be placed through the tympanic membrane.

In another refinement, the fluid-filled tube-shaped device is used for transmitting hearing information where the fluid-filled tube-shaped device traverses the tympanic membrane.

In yet another aspect, a method for implanting the semi-implantable hearing device set forth herein in a subject is provided. The method includes a step of placing the implantable semi-implantable hearing device of claims 1 to 25 in a subject’s ear or anatomical structure adjacent to the subject’s ear by surgically placing the replaceable outer ear unit in the subject.

In yet another aspect, the method further includes a step of placing the replaceable outer ear unit in the subject’s ear canal.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1. Schematic of a semi-implantable hearing device.

FIG. 2A. Schematic of a fluid-filled tube-shaped device.

FIG. 2B. Schematic of a fluid-filled tube-shaped device attached to an ear structure.

FIG. 2C. Schematic of a fluid-filled tube-shaped device attachable with magnets.

FIG. 2D. Schematic of a fluid-filled tube-shaped device with heat-activated metal arms.

FIG. 3A. Schematic of a semi-implantable hearing device that transfers energy from a replaceable outer ear unit to a fluid-filled tube-shaped device.

FIG. 3B. Schematic of a voltage to current circuit that can be used in the semi-implantable hearing device of FIGS. 3A, 4A-C, and 5.

FIG. 4A. Schematic of replaceable outer ear unit having a transmitter coil and a fluid-filled tube-shaped device having a receiver coil.

FIG. 4B. Schematic of a fluid-filled tube-shaped device having a receiver coil with ends attached to a resistor.

FIG. 4C. Schematic of a fluid-filled tube-shaped device having a receiver coil with ends attached to a piezoelectric component.

FIG. 5. Schematic of a semi-implantable hearing device that transfers energy via light from a replaceable outer ear unit to a fluid-filled tube-shaped device.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4. ... 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ± 0%, 0.1%, 0.5%, 1%, 2%, 3%, 4%, 5% or 10% of the value or relative characteristic.

The term “one or more” means “at least one” and the term “at least one” means “one or more.” The terms “one or more” and “at least one” include “plurality” as a subset.

The term “electrical communication” means that an electrical signal is either directly or indirectly sent from an originating electronic device to a receiving electrical device. Indirect electrical communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electrical communication can be accomplished with wired components, wirelessly connected components, or a combination thereof.

The term “electrical signal” refers to the electrical output from an electronic device or the electrical input to an electronic device. The electrical signal is characterized by voltage and/or current. The electrical signal can be stationary with respect to time (e.g., a D.C. signal) or it can vary with respect to time.

The term “electronic component” refers is any physical entity in an electronic device or system used to affect electron states, electron flow, or the electric fields associated with the electrons. Examples of electronic components include, but are not limited to, capacitors, inductors, resistors, thyristors, diodes, transistors, etc. Electronic components can be passive or active.

The term “electronic device” or “system” refers to a physical entity formed from one or more electronic components to perform a predetermined function on an electrical signal.

It should be appreciated that in any figures for electronic devices, a series of electronic components connected by lines (e.g., wires) indicates that such electronic components are in electrical communication with each other. Moreover, when lines directed connect one electronic component to another, these electronic components can be connected to each other as defined above.

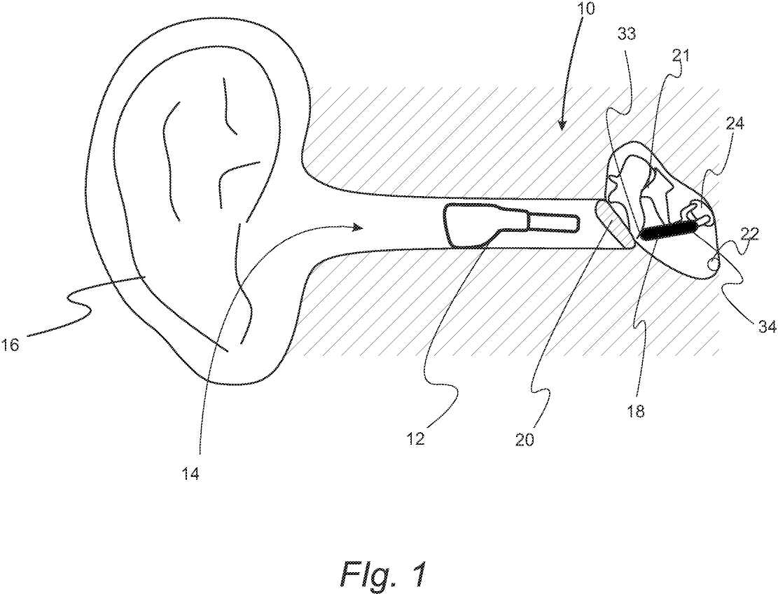

With reference to FIG. 1, a totally or partially concealed, partially implantable hearing device is provided. Implantable hearing device 10 includes a replaceable outer ear unit 12 having a means for generating energy responsive to acoustic energy received by the replaceable outer ear unit. The replaceable outer ear unit 12 is adapted to be located inside a subject’s outer ear canal 14 or behind a subject’s ear 16. The implantable hearing device also includes a fluid-filled tube-shaped device 18 that is placed in the middle ear, mastoid, or other structure proximate to the middle ear. The tympanic membrane is labeled with item number 20, the ossicular chain as item number 21, the round window as item 22, and the oval window as 24. In a refinement, fluid-filled tube-shaped device 18 can be placed proximate to the inner ear. Characteristically, the fluid-filled tube-shaped device 18 is adapted to receive transferred energy from the replaceable outer ear unit 12 such that energy received by the fluid-filled tube-shaped device is transferred to the inner ear via fluid contained in the fluid-filled tube-shaped device. In a refinement, the replaceable outer ear unit 12 is hermetically sealed and implanted in a subject’s body.

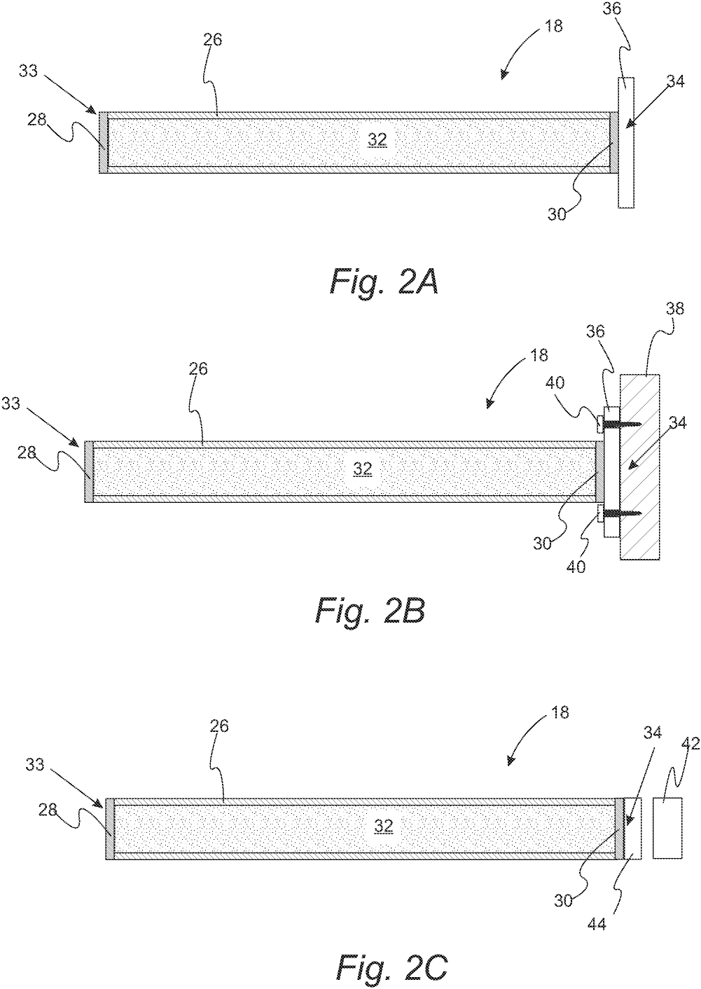

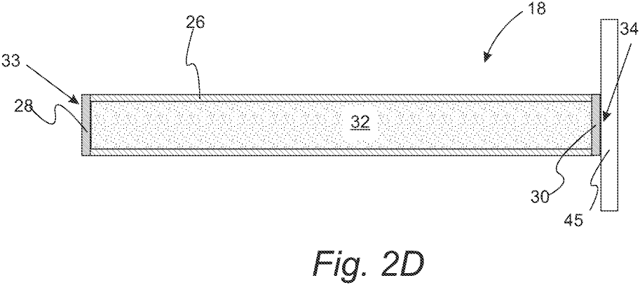

FIGS. 2A, 2B, and 2C provide cross-sections of examples for fluid-filled tube-shaped device 18. Fluid-filled tube-shaped device 18 includes a closed flexible tube 26 formed from a flexible tube 26 closed with end caps 28 and 30. In a refinement, one or both of end caps 28 and 30 are monolithic with tube 26. Therefore, closed flexible tube 26 has an outer end 33 and an inner end 34. Fluid 32 is contained within closed flexible tube 26. Fluid 32 can completely fill or partially fill closed flexible tube 26. In a refinement, closed flexible tube 26 is composed of a rubber or flexible plastic such as silicone rubber. In another refinement, the liquid 32 in the tube is selected from the group consisting of fluid silicone, silicone gel, saline, water, and compositions thereof. In another refinement, the liquid 32 in the tube can include photo-responsive compositions such as photo-responsive dye-doped liquid crystals. Fluid-filled tube-shaped device 18 also includes an attachment flange 36 attached in an end of closed flexible tube 26. FIG. 2B depicts the fluid-filled tube-shaped device 18 attached to ear structure 38 with screws or bolts 40. The contact of filled tube-shaped device 18 with the inner ear contact can be rigid or flexible. In a refinement, the fluid-filled tube-shaped device 18 is secured using flanges. In another refinement, the fluid-filled tube-shaped device 18 the fluid-filled tube-shaped device is secured using screw(s). As depicted by FIG. 2C, fluid-filled tube-shaped device 18 is secured to a subject by attaching a magnet 42 to the ossicular chain 21 in the ear. A mating magnet or ferromagnet metal 44 can be adhered to the inner end 34 of closed flexible tube 26. In yet another refinement, fluid-filled tube-shaped device 18 is secured in place using silicone or metal. In still another refinement as depicted in FIG. 2D, the fluid-filled tube-shaped device is secured using heat-activated metal arms 45. An example of such heat activated arms are composed of nitinol (i.e., nickel titanium) arms that can be heated with a laser thereby changing shape and secure the implant to the middle ear structures.

Although fluid-filled tube-shaped device 18 can be placed in the middle ear or mastoid, it can also be positioned in other structures related to hearing. In a refinement, fluid-filled tube-shaped device 18 makes contact with the tympanic membrane. In a further refinement, fluid-filled tube-shaped device 18 makes contact with the tympanic membrane at the outer end 33 of closed flexible tube 26. In another refinement, fluid-filled tube-shaped device 18 makes contact with outer end 33 under the skin of the subject’s ear canal. In still another refinement, fluid-filled tube-shaped device 18 makes outer contact with outer end 33 under the skin behind or above the ear. In another refinement, fluid-filled tube-shaped device 18 contacts the inner ear contact at the round window membrane or the oval window. In a further refinement, fluid-filled tube-shaped device 18 contacts the inner ear contact at the round window membrane or the oval window with the inner end 34. It should also be appreciated that fluid-filled tube-shaped device 18 can be used for transmitting hearing information where the fluid-filled tube-shaped device can be placed through the tympanic membrane. In a refinement, the fluid-filled tube-shaped device is used for transmitting hearing information where the fluid-filled tube-shaped device traverses the tympanic membrane.

It should also be appreciated that fluid-filled tube-shaped device 18 can be placed in the middle ear by any number of surgical procedures known in the art. For example, fluid-filled tube-shaped device 18 can be used for transmitting hearing information where inner ear contact is made with the inner ear through a surgically or disease-created opening into the inner ear.

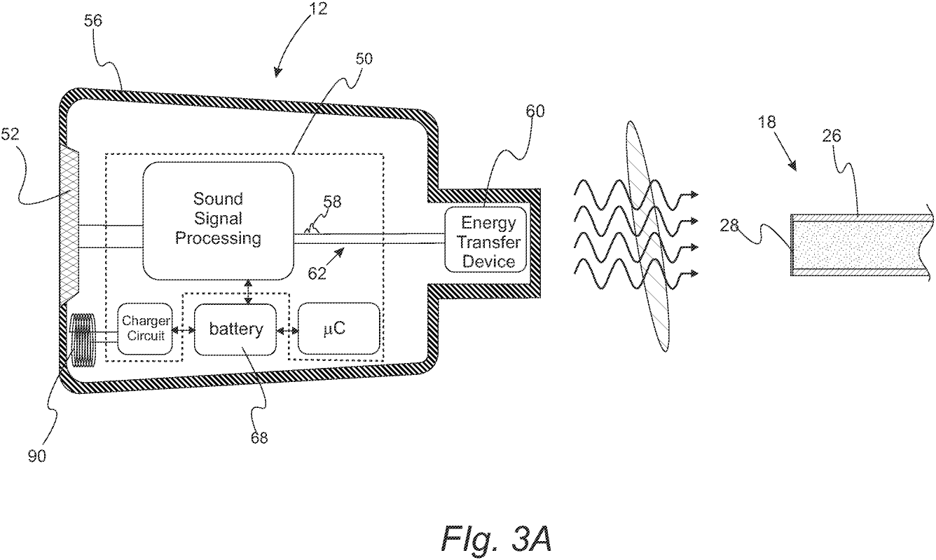

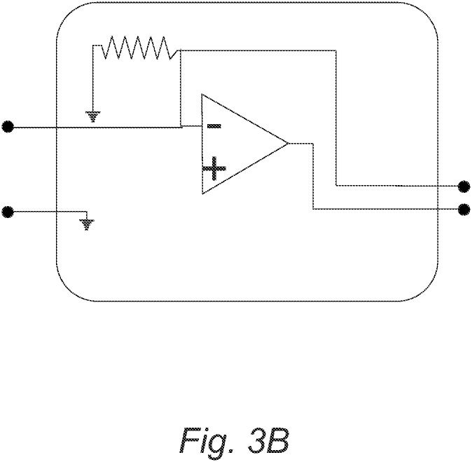

FIG. 3A provides a schematic of replaceable outer ear unit 12. Replaceable outer ear unit 12 includes circuitry 50, which includes one or more elements for signal processing, recharging, programming, and additional functions of the hearing aid device. Microphone 52 is located at the end of replaceable outer ear unit 12. When replaceable outer ear unit 12 is placed in the ear canal, as depicted in FIG. 1, microphone 52 faces the entrance of the ear canal when worn. Sound is received by microphone 52 and the signal process circuitry of circuitry 50 processes the received sound signal. The signal process circuitry of circuitry 50 can include amplifiers, voltage to current circuits as depicted in FIG. 3B, pulse-width modulation (PWM) circuits, or pulse-duration modulation (PDM) circuits, and the like. PWM circuits and/or or PDM are particularly useful for light coupling as described in international patent Appl. No. WO2009155358A1; the entire disclosure of which is hereby incorporated by reference. In another variation particularly suitable for optical coupling, circuitry 50 can incudes a pulse generator so that light pulses modulated by the sound signal are transmitted. In another refinement, circuitry 50 can includes a control component such as a microprocessor or any control component for control the sound signal processing. Microphone 42 is located at the end of outer component 20. Details for components of circuitry 50 are found in U.S. Pat No. 9407994B2; the entire disclosure of which is hereby incorporated by reference. Replaceable outer ear unit 12 includes housing 56, which provides one or more features to hold the hearing aid device comfortably in place as described by U.S. Pat No. 9407994B2; the entire disclosure of which is hereby incorporated by reference. Replaceable outer ear unit 12 can include one or more elements to reduce sound from reaching one or more portions of the ear canal, tympanic membrane, middle ear, or inner ear. In one refinement, housing 56 may completely seal the ear canal and prevent natural sound from reaching the tympanic membrane. In another refinement, housing 56 can be designed with baffles to impede sound from reaching the tympanic membrane while allowing the pressure to equalize between both sides of the device. Housing 56 may be designed to have no sealing (non-occluding) and allow free passage of sound. Housing 56 can also be designed to be flexible to allow relative movements between components to allow the device to better conform to the ear canal. Housing 56 can be composed of a polymer and in particular, a soft polymer or plastic. The output signal 58 from circuitry 50 is carried to energy transfer device 60 through tympanic membrane 20 via wires 62. The energy transfer device 60 transfers energy to fluid-filled tube-shaped device 18, which responds in a manner to allow the perception of sound in a patient. This response typically involves movement and/or time dependent vibrations in the closed flexible tube 26. The energy transfer device 60 transfers energy by electromagnetic coupling (e.g., inductive coupling and radiofrequency coupling) and optical coupling as set forth below in more detail. The energy transfer device 60 transfers energy by electromagnetic coupling (e.g., inductive coupling and radiofrequency coupling), direct wiring, and optical coupling as set forth below in more detail.

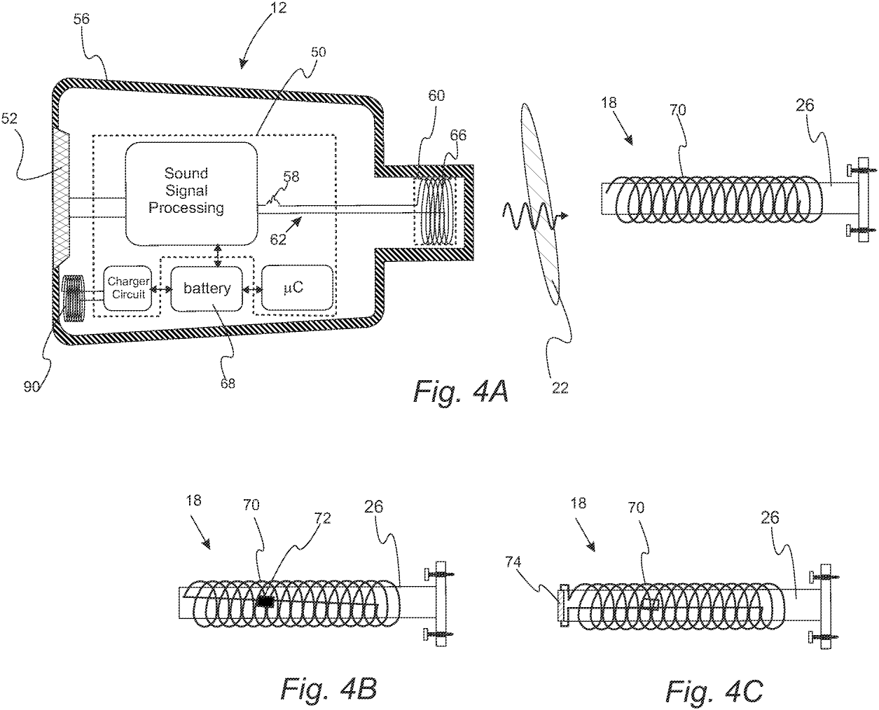

FIGS. 4A, 4B, and 4C depict variations in which energy is transferred from the replaceable outer ear unit 12 to the fluid-filled tube-shaped device 18 using electromagnetic energy, and in particular, by stimulation by electromagnetic waves (e.g., radiofrequency stimulation). In this variation, energy transfer device 60 includes a coil that can be coupled (e.g., inductively coupled) to the fluid-filled tube-shaped device 18. As set forth above, replaceable outer ear unit 12 includes circuitry 50, which includes one or more elements for signal processing, recharging, programming, and additional functions of the hearing aid device. Microphone 52 is located at the end of replaceable outer ear unit 12. When replaceable outer ear unit 12 is placed in the ear canal, as depicted in FIG. 1, microphone 52 faces the entrance of the ear canal when worn. Sound is received by microphone 52 and circuitry 50 processes the received sound signal. Replaceable outer ear unit 12 includes housing 56, which provides one or more features to hold the hearing aid device comfortably in place. Replaceable outer ear unit 12 can include one or more elements to reduce sound from reaching one or more portions of the ear canal, tympanic membrane, middle ear, or inner ear. In one refinement, housing 56 may completely seal the ear canal and prevent natural sound from reaching the tympanic membrane. The details of housing 56 are set forth above. The output signal 58 from circuitry5 is carried to the energy transfer device 60 via wires 62. Energy transfer devices 60 includes transmitter coil 66 which is in electrical communication with circuitry 50. Transmitter coil 66 transfers energy to fluid-filled tube-shaped device 18 through the tympanic membrane 20, which responds in a manner to allow the perception of sound in a patient. This response typically involves movement and/or vibrations in the closed flexible tube 26 derived from output signal 58. In a refinement, the radio frequency stimulation provided to closed flexible tube 26 is in the range 20 kHz to around 300 GHz. In should be appreciated that electromagnet waves having a frequency less than 20 kHz and greater that 300 GHz can also be used.

Still referring to FIGS. 4A, 4B, and 4C, in a refinement, fluid-filled tube-shaped device 18 includes receiver 70 coil wound around closed flexible tube 26. In a further refinement, transmitter coil 66 is aligned with receiver coil 70 to maximize inductive coupling and/or transfer of energy to closed flexible tube 26. In a refinement, the ends of receiver coil 70 is attached to closed flexible tube 26. In another refinement, the ends of receiver coil 70 are electrically connected together. In a further refinement, a resistor and/or capacitor 72 is connected between the ends of receiver coil 70. In still another refinement, a piezoelectric component 74 is connected between the ends of receiver coil 70 and optionally, adhered to closed flexible tube 26. Piezoelectric component 74 can be composed of or includes a layer of a piezoelectric material. Examples of piezoelectric materials includes langasite (La₃Ga₅SiO₁₄) gallium orthophosphate (GaPO₄), lithium niobate (LiNbO₃), lithium tantalate (LiTaO₃), quartz, berlinite (AlPO₄), lead titanate (PbTiO₃), lead zirconate titanate (Pb[ZrₓTi₁₋ₓ]03 with 0 ≤ x ≤ 1), potassium niobate (KNbO₃), sodium tungstate (Na₂WO₃), Ba₂NaNb₅O₅, Pb₂KNb₅O₁₅, zinc oxide (ZnO), and combinations thereof.

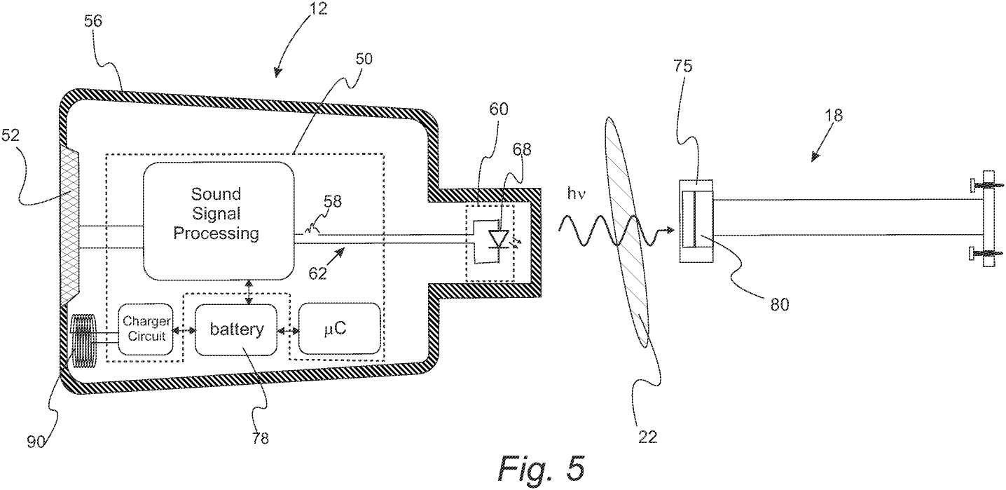

FIG. 5 depicts a variation in which energy is transferred from the replaceable outer ear unit 12 to the fluid-filled tube-shaped device 18 using light. In this variation, energy is transferred from the replaceable outer ear unit 12 to the fluid-filled tube-shaped device 12 via a light-based transmission. In a refinement, energy is transferred from the replaceable outer ear unit 12 to the fluid-filled tube-shaped device 12 via a light-based transmission and piezoelectric translation. Moreover, energy transfer device 60 includes a light source (e.g., a photodiode or a laser diode). As set forth above, replaceable outer ear unit 12 includes circuitry 50, which includes one or more elements for signal processing, recharging, programming, and additional functions of the hearing aid device. Microphone 52 is located at the end of replaceable outer ear unit 12. When replaceable outer ear unit 12 is placed in the ear canal, as depicted in FIG. 1, microphone 52 faces the entrance of the ear canal when worn. Sound is received by microphone 52 and circuitry 50 processes the received sound signal. Replaceable outer ear unit 12 includes housing 56, which provides one or more features to hold the hearing aid device comfortably in place. Replaceable outer ear unit 12 can include one or more elements to reduce sound from reaching one or more portions of the ear canal, tympanic membrane, middle ear, or inner ear. In one refinement, housing 56 may completely seal the ear canal and prevent natural sound from reaching the tympanic membrane. The details of housing 56 are set forth above. The output signal 58 from circuitry 50 is carried to energy transfer device 60 via wires 62. Energy transfer devices 60 includes light source 68 which is in electrical communication with circuitry 50. Light sources include any device capable of receiving the electronic drive signal and producing a light output of suitable frequency, intensity, and modulation. Examples of light sources include, but are not limited to, light emitting diodes, semiconductor lasers, and the like. Light source 68 transfers energy to fluid-filled tube-shaped device 18 through tympanic membrane 20. Fluid-filled tube-shaped device 18 responds in a manner to allow the perception of sound in a patient. In a refinement, fluid-filled tube-shaped device 18 includes a light detection device 75 which receive the light and transfers energy to the fluid in closed flexible tube 26 causing movement (e.g., time dependent vibration) therein that a patient can interpret as sound. In another refinement, photo-responsive device 75 includes a photocell (e.g., a photosensor diode or solar cell).

In a variation, light detection device 75 includes a photo responsive device 80. In another refinement, photo responsive device 80 includes photostrictive materials (e.g., PLZT) are described in U.S. Pub. No. 2006/0189841; the entire disclosure of which is hereby incorporated by reference. In general, the photostrictive effect is a light-induced-nonthermal dimension changes of materials. Since the photostrictive materials are most active a certain wavelengths of light, light source 68 is selected to output light at such wavelengths. In a refinement, photo responsive device 80 includes one or more layers (1 to 5) of photostrictive materials typically coated onto an optional substrate. Details of photo responsive devices 80 that include photostrictive materials which can be used in the present variations are set forth in U.S. Pat No. 7867160; the entire disclosure of which is hereby incorporated by reference in its entirety.

In a refinement, the light input to photo-responsive device 80 provides sufficient power to produce mechanical vibrations such that fluid-filled tube-shaped device 18 couples to a subject’s hearing transduction pathway. This couple can induce neural impulses in the subject that is interpreted as sound. Since the photostrictive materials are most active certain wavelengths of light, light source 68 is selected to output light at such wavelengths.

Referring to FIGS. 3, 4, and 5, battery 78 can power semi-implantable hearing device 10. In a refinement, charging interface 90 is used to recharge battery 68. In a refinement, charging interface 90 is a recharge coil 80 that can recharge battery 68 via inductive coupling to an external power source. In another refinement, charging interface 90 is a photocell. In still another refinement, charging interface 90 is an electronic connector that allows charging with an external power source. In some refinements, recharging of battery 68 is not a requirement for successful implementation of the device.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A semi-implantable hearing device comprising:
a replaceable outer ear unit having an energy transfer device for generating energy responsive to acoustic energy received by the replaceable outer ear unit, the replaceable outer ear unit being adapted to be located inside a subject’s outer ear canal or behind a subject’s ear; and
a fluid-filled tube-shaped device that is placed in the middle ear or mastoid, the fluid-filled tube-shaped device adapted to receive transferred energy from the replaceable outer ear unit wherein energy received by the fluid-filled tube-shaped device is transferred to a subject’s inner ear via fluid contained in the fluid-filled tube-shaped device.

2. The semi-implantable hearing device of claim 1, wherein the fluid-filled tube-shaped device includes the fluid enclosed by a closed flexible tube.

3. The semi-implantable hearing device of claim 1, wherein the energy is transferred from the replaceable outer ear unit to the fluid-filled tube-shaped device using electromagnetic stimulation.

4. The semi-implantable hearing device of claim 1, wherein energy is transferred from the replaceable outer ear unit to the fluid-filled tube-shaped device via a radiofrequency stimulation.

5. The semi-implantable hearing device of claim 1, wherein energy is transferred from the replaceable outer ear unit to the fluid-filled tube-shaped device via inductive coupling.

6. The semi-implantable hearing device of claim 1, wherein the replaceable outer ear unit is configured to transfer energy to the fluid-filled tube-shaped device via a light-based transmission and piezoelectric translation.

7. The semi-implantable hearing device of claim 1, wherein the replaceable outer ear unit is configured to transfer energy to the fluid-filled tube-shaped device via aligned coils such that a first coil in the replaceable outer ear unit is aligned with a second coil in the fluid-filled tube-shaped device.

8. The semi-implantable hearing device of claim 1, wherein the fluid-filled tube-shaped device includes an attached piezoelectric component.

9. The semi-implantable hearing device of claim 1, wherein the fluid-filled tube-shaped device includes an attached photo-responsive device that includes a photostrictive material.

10. The semi-implantable hearing device of claim 1, wherein the fluid-filled tube-shaped device is secured by attaching the fluid filled tube to the ossicular chain in the subject’s ear.

11. The semi-implantable hearing device of claim 1, wherein the replaceable outer ear unit is hermetically sealed and implanted in a subject’s body.

12. The semi-implantable hearing device of claim 1, wherein the fluid-filled tube-shaped device is configured to make inner ear contact by contacting the round window membrane.

13. The semi-implantable hearing device of claim 1, wherein the fluid-filled tube-shaped device is configured to make inner ear contact by contacting the oval window or one or more ossicle.

14. The semi-implantable hearing device of claim 1, wherein the fluid-filled tube-shaped device is configured to make inner ear contact through a surgically or disease-created opening into the inner ear.

15. The semi-implantable hearing device of claim 1, wherein the fluid-filled tube-shaped device is configured to make outer contact with the tympanic membrane.

16. The semi-implantable hearing device of claim 1, wherein the fluid-filled tube-shaped device is configured to make outer contact under skin of the subject’s ear canal.

17. The semi-implantable hearing device of claim 1, wherein the fluid-filled tube-shaped device is configured to make outer contact under skin behind or above the ear.

18. The semi-implantable hearing device of claim 1, wherein the fluid-filled tube-shaped device is configured to transmit hearing information with a rigid inner ear contact.

19. The semi-implantable hearing device of claim 1, wherein the fluid-filled tube-shaped device transmits hearing information with a flexible inner ear contact.

20. The semi-implantable hearing device of claim 1, wherein the fluid-filled tube-shaped device is used for transmitting hearing information where the fluid-filled tube-shaped device can be placed through the tympanic membrane.

21. The semi-implantable hearing device of claim 1, wherein the fluid-filled tube-shaped device is used for transmitting hearing information where the fluid-filled tube-shaped device traverses the tympanic membrane.

22. The semi-implantable hearing device of claim 1, wherein the fluid-filled tube-shaped device is used for transmitting hearing information where the fluid-filled tube-shaped device is secured using flanges.

23. The semi-implantable hearing device of claim 1, wherein the fluid-filled tube-shaped device is used for transmitting hearing information where the fluid-filled tube-shaped device is secured using screw(s).

24. The semi-implantable hearing device of claim 1, wherein the fluid-filled tube-shaped device is used for transmitting hearing information where the fluid-filled tube-shaped device is secured using heat-activated metal arm(s).

25. The semi-implantable hearing device of claim 1, wherein the fluid-filled tube-shaped is secured in place using silicone or metal.
